(19) United States
(12) Patent Application Publication
(10) Pub. No.: US 2014/0219064 A1
(43) Pub. Date: Aug. 7, 2014

(54) MEDICAL APPARATUS FOR DATA STORAGE

(71) Applicants: Joseph C. Filipi, Aliso Viejo, CA (US); Chuck J. Filipi, Omaha, NE (US); Wayne W. Hohman, Round Rock, TX (US)

(72) Inventors: Joseph C. Filipi, Aliso Viejo, CA (US); Chuck J. Filipi, Omaha, NE (US); Wayne W. Hohman, Round Rock, TX (US)

(21) Appl. No.: 14/170,481

(22) Filed: Jan. 31, 2014

Related U.S. Application Data
(60) Provisional application No. 61/849,617, filed on Jan. 31, 2013.

(51) Int. Cl. A61B 5/00; G04G 21/00

(52) U.S. Cl. CPC A61B 5/742; G04G 21/00
USPC 368/10

(57) ABSTRACT

An electronic apparatus and method for storing patient data, information, or records and/or notifying a patient of a medical activity to be performed. The electronic apparatus may be wearable, such as a watch or other jewelry, or may be non-wearable, such as a household appliance or other equipment. The electronic apparatus may include a screen, a processor, a memory, and an input/output component, such as a USB plug or socket, for allowing outside devices to connect with the electronic apparatus and access the memory and/or the processor. The electronic apparatus may communicate with remote devices to transmit patient data or compliance information. Gaming functionality, such as one assigning points to a patient based upon compliance, may be used to encourage greater compliance and/or foster competition between patients. Prizes may be selected or awarded to a patient based upon the assigned points.

### CROSS REFERENCE TO RELATED APPLICATIONS

This application claims the benefit of U.S. Provisional Patent Application Ser. No. 61/849,617, filed on Jan. 31, 2013, entitled “THE LIFE WATCH,” which is hereby incorporated by reference in its entirety.

### BACKGROUND

1. Field of the Invention

The present invention is generally related to a medical information and/or medication administration management system and method. More particularly, the present invention relates to a watch or other electronic apparatus to a patient whereby medical personnel can access data or information, such as information vital to the patient's health, that is stored on the watch or other electronic apparatus.

2. Description of the Related Art

Medication and healthcare information management is clumsy and incongruous. We intend to transform the way information is held by the patient and transported between caregivers.

The current medical system sees a vast number of re-admissions (e.g., 30% of patients) to the hospital due to medication management errors in the post hospital setting. By cutting this percentage in half, billions of dollars in unnecessary re-admission costs and countless lives could be saved annually. The current medical system also fails to adequately provide a manner for information to follow the patient between care givers and/or to be readily accessible in an organized fashion to both parties as needed. As of October 2013, Medicare has been penalizing providers when preventable readmissions to the hospital occur within 30 days after discharge.

It has been shown that readmissions are a costly component of Medicare-covered hospital services, with MedPAC reporting that readmissions within 30 days accounted for $15 billion of Medicare spending.” These previously paid out amounts are expected to eventually fall on the shoulders of the healthcare providers, having a major and possibly catastrophic impact on hospitals.

In addition to readmission rates, healthcare efficiency can also be impacted via improved record keeping systems that track and/or store patient data. A multitude of medical record systems have been developed and utilized, but have not shown significant benefit to patients. For example, these have included hand written records both in the hospital, the pharmacy, and the doctor's office. More recently,; however, even those records continue to be discoordinate due to significant interface difficulties when patient's transfer between healthcare systems, hospitals, or even different doctors.

An ideal system or method would aid in reducing the rate at which readmissions to hospitals within the first 30 days after discharge occur, particularly in light of increased emphasis on healthcare reform and/or insurance companies looking to cut costs, potentially saving the provider and/or insurer millions of dollars. An ideal system or method would also or alternatively aid in improving healthcare efficiency by improving the storing and/or transferring of patient data or records throughout the patient's care.

### SUMMARY

One purpose of the present invention to develop support systems, such as a personal electronic device (e.g., a watch) that is capable of storing data and easily being programmed by a user to operate with such data or in response to particular user desires. In one embodiment, a call center may be utilized in addition to, or in replacement of, the personal electronic device, for example to answer patient questions and/or proactively make phone calls or send messages (e.g., SMS text messages) to family members in regards to a patient. Such messages may be sent, for example, if a patient misses a particular medication schedule. The personal electronic device and/or the call center can help remind patients about their next medication, for example with a simple alarm or via direct personal contact. The personal electronic device may include a wrist band that lights up and/or may include a USB port built in for facilitating access to the data stored therein and/or to provide access to the programmable functions of the device.

In other embodiments, the present invention may be designed to sound an alarm on the electronic device that signifies the time a medication is due with the name of the drug scrolling across a screen or face of the device. For example, if a patient does not remove the medication from a corresponding electronic pillbox, a message may be sent to a designated family member or other individual. The opening and emptying of the contents of the electronic pillbox may reset the watch and/or turn off the alarm.

While one purpose of the present invention is medication management, diabetics may also have the opportunity to program an alarm in order to check their blood sugar at regular intervals. Gamification principles and competition may be used to drive patients toward higher compliance. For example, once a patient opens their pillbox and empties the contents, a message can be transmitted to a remote location (e.g., the primary care physician's office) indicating compliance. Such compliance can result in points awarded to the patient. At the end of the designated time period, “winners” are chosen based on their compliance and/or points, which can result in an award for the patient.

Still other embodiments may facilitate, for example, in an emergency department, the patient's clinical pathway to be chosen as quickly as possible. As this pathway is determined by the physician's expertise and the information at the time at hand, increased knowledge about the patient's current condition, past conditions, and/or a variety of other patient information can be used to improve the diagnosis. The provision of such information may be included as part of the electronic device. For example, using embedded menus, a patient may enter their pertinent medical history (e.g., in bullet point fashion for ease of use from the physician) via connecting the electronic device to a smart phone, tablet, personal computer, laptop, etc. via a user interface component, such as a USB port. Thus, once the patient arrives in the hospital setting, they can provide the care giver with the electronic device and the care giver may easily plug it into other components available at the hospital, such as any desktop computer. This increased ease in making patient data available can shorten the time between admission and the medical diagnosis, potentially saving both time and lives.

In yet another embodiment, an electronic device may have a deeper structural support. For example, once the patient arrives in the emergency room, Bluetooth technology embedded in the device may trigger a chain of events linking the hospital based computer to a central data warehouse where the patients' medical history is stored.

The present invention is related to a method and system for storing patient data and/or reminding a patient of medical activity to be performed. In one embodiment, an electronic apparatus for a patient may include a band configured to be worn around a wrist of the patient, a processor coupled with the band, and a screen coupled with the band and the processor, wherein the screen is configured to display a message relating to a medical activity to be performed by the patient.

In another embodiment, a medication notification system may include a watch having a wristband, a speaker, a display, and a processor connected with the display, the processor configured to cause the speaker to alarm based on a schedule for consumption of a medication. A pillbox having a processor may be configured to interface with the processor of the watch and wherein the speaker ceases to alarm based on at least a portion of the pillbox being emptied.

In still another embodiment, a method for encouraging compliance of a patient for a medical activity may include providing a watch to the patient, programming the watch to notify the patient about the medical activity, notifying the patient about the medical activity via the watch, determining whether the patient complied with the notifying of the patient about the medical activity, and transmitting information corresponding to whether the patient complied with the notifying of the patient about the medical activity.

### BRIEF DESCRIPTION OF THE DRAWINGS

The features, objects, and advantages of the present invention will become more apparent from the detailed description set forth below when taken in conjunction with the drawings, wherein:

FIG. 1A shows a plurality of electronic apparatuses for storing patient data and/or reminding a patient of medical activity to be performed according to one embodiment of the present invention;

FIG. 1B shows a side view of an electronic apparatus for storing patient data and/or reminding a patient of medical activity to be performed according to one embodiment of the present invention;

FIG. 2A shows a first screen of a software program configured to interface with an electronic apparatus for storing patient data and/or reminding a patient of medical activity to be performed according to one embodiment of the present invention;

FIG. 2B shows a second screen of a software program configured to interface with an electronic apparatus for storing patient data and/or reminding a patient of medical activity to be performed according to one embodiment of the present invention;

FIG. 2C shows a third screen of a software program configured to interface with an electronic apparatus for storing patient data and/or reminding a patient of medical activity to be performed according to one embodiment of the present invention;

FIG. 2D shows a fourth screen of a software program configured to interface with an electronic apparatus for storing patient data and/or reminding a patient of medical activity to be performed according to one embodiment of the present invention;

FIG. 2E shows a fifth screen of a software program configured to interface with an electronic apparatus for storing patient data and/or reminding a patient of medical activity to be performed according to one embodiment of the present invention;

FIG. 2F shows a sixth screen of a software program configured to interface with an electronic apparatus for storing patient data and/or reminding a patient of medical activity to be performed according to one embodiment of the present invention;

FIG. 3 shows a front view of an electronic apparatus being worn by a patient that is configured to store patient data and/or remind the patient of medical activity to be performed according to one embodiment of the present invention;

FIG. 4 shows a portable electronic receiver that is configured to interface with an electronic apparatus that is configured to store patient data and/or remind a patient of medical activity to be performed according to one embodiment of the present invention;

FIG. 5 shows a pillbox configured to remotely interface with an electronic apparatus that is configured to store patient data and/or remind the patient of medical activity to be performed according to one embodiment of the present invention;

FIG. 6 shows a sensor for detecting if at least a portion of a pillbox has been emptied, the pillbox configured to remotely interface with an electronic apparatus that is configured to store patient data and/or remind the patient of medical activity to be performed according to one embodiment of the present invention; and

FIG. 7 shows a collection of data relating to patient compliance for the purposes of gamification of the use of an electronic apparatus that is configured to store patient data and/or remind a patient of medical activity to be performed according to one embodiment of the present invention.

### DETAILED DESCRIPTION

The detailed description of exemplary embodiments herein makes reference to the accompanying drawings and pictures, which show the exemplary embodiment by way of illustration and its best mode. While these exemplary embodiments are described in sufficient detail to enable those skilled in the art to practice the invention, it should be understood that other embodiments may be realized and that logical and mechanical changes may be made without departing from the spirit and scope of the invention. Thus, the detailed description herein is presented for purposes of illustration only and not of limitation. For example, the steps recited in any of the method or process descriptions may be executed in any order and are not limited to the order presented. Moreover, any of the functions or steps may be outsourced to or performed by one or more third parties. Furthermore, any reference to singular includes plural embodiments, and any reference to more than one component may include a singular embodiment.

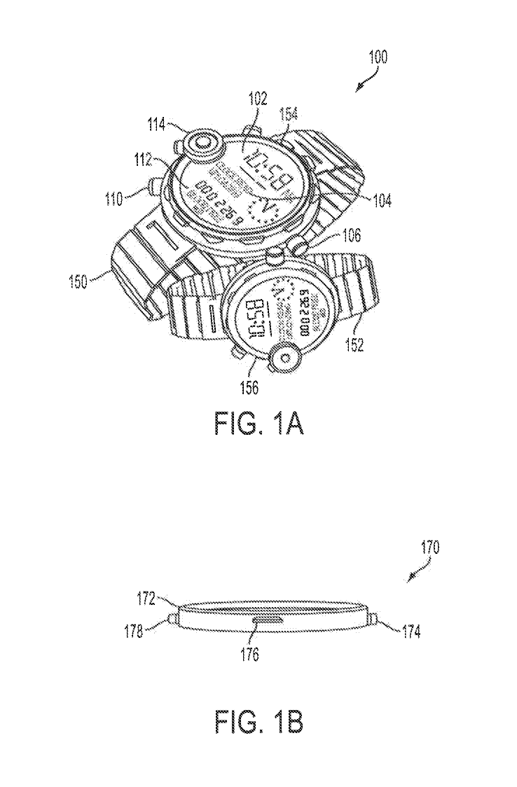

Referring first to FIG. 1, a plurality of watches 100 including watchbands (150, 152) and screens (154, 156) are shown. The screen (154, 156) may contain information that will electronically demonstrate one of more pieces of information or data at a time desired. For example, the one or more pieces of information or data may include: 1) name and dose of medication to be taken, 2) time, 3) day of week, and/or 4) watch status. Moreover, in certain embodiments, may additionally or alternatively be 5) an alarm override reset button, 6) changing functions button 7) Blue-tooth technology for communication.

In one embodiment, one or both of the plurality of watches 100 may include a radio frequency identification (RFID) tag, for example, to assist in the acquisition of medical information including the positive identification of the particular patient carrying one of the plurality of watches 100. In certain embodiments, one or both of the plurality of watches 100 may include a mini-USB (Universal Serial Bus) port (male or female) that will allow transfer of data, information, or patient records between devices. These data, information, or patient records may include EKG's, medications, the most recent history and physical, CAT scan impressions, allergies, etc. These data, information, or patient records may also include medication administration times that can be input (e.g., by the consumer on a desktop computer and/or by a physician) to help aid in medication compliance. In another embodiment, the data, information, or patient records may be configured to be transported from the facility to an information warehouse (e.g., a remote server) or vice versa with the assistance of hospital personnel or a separate network of technologists specializing in the electronic transfer of such information.

The plurality of watches 100 may be a male watch and a female watch. Each of the plurality of watches 100 may include features or components that are the same or similar to one another. The screen 154 may be configured to display a day of the week with time 102 and a status message 104 (e.g., a message indicating whether the watch is uploading/downloading data, in a programming mode, etc.) The screen 154 may also display to a user a medication to be taken 112. Certain of these display functions, modes, and/or information may be manipulated by a user via a changing functions button 106. If a speaker is sounding an alarm or the display 154 visually indicating an alarm or message to a user, an alarm override button 110 may be manipulated by the user to end the alarm. In certain embodiments, for example, if a watch is configured to communicate with remote devices, such as a pillbox, via Bluetooth technology, a Bluetooth portion 114 of the watch may be included that includes the necessary Bluetooth components for proper operation.

Each of the plurality of watches 100 may be configured to store data, information, and/or a patient record upon memory contained therein. This data, information, and/or patient record may be interacted with (e.g., viewed, updated, etc.) via a USB connection. Thus, by plugging the watch via the USB port (male or female plug) into an external device or system, such as a desktop PC, access to the memory may be made available. An alternative embodiment may use other manners of allowing data access (e.g., other types of plugs or sockets and/or wireless communication).

In operation, a user would first program the watch to adequately alarm at specific times according to a schedule of desired medical activity. The user could either be a particular patient that would be using the watch daily or may be a physician or other care giver. In one example, the medical activity may be particular times in which particular medications are meant to be taken by a patient. Thus, the watch acts as a reminder to help ensure compliance with the patient's medication regime. This programming may be done wirelessly, via direct or wired connection with an external device, and/or using buttons/knobs/etc. located upon the watch itself.

After programming and during use, a processor of the watch will determine when the particular time has occurred to notify the patient of a particular medical activity, for example, a time when a certain medication is to be taken. Once determined, the processor of the watch will interface with a speaker, screen, and/or other components of the watch to notify the patient. For example, a speaker of the watch may emit an audible sound or alarm, a screen of the watch may display a scrolling message, such as the name and/or dosage of medication to take, a watchband may illuminate in different colors, etc. In certain embodiments, the notification to the patient may be selectable, customizable, and/or personalized to the particular patient. For example, the notification may be an audible sound or alarm that speaks words of affirmation and incentive in order to increase the incentive of the patient to comply with the notification (e.g., a member of the patient's family may record a personalized message to the patient that speaks personally to the patient to inform him/her that it is time to perform the medical activity and/or congratulates the patient upon complying with a previous notification). Many other personalized notifications may be used in alternative embodiments, such as personal text messages, favorite songs, favorite colors being displayed, etc. Other notifications may be incorporated in addition or alternative to those discussed (e.g., vibration).

Upon being notified, the patient responds by performing the medical activity being designated by the watch. In the case of a medication reminder, the patient takes the particular medication in the dosage being identified by the processor of the watch. After performing the medical activity, the patient may turn off the notifications currently being output by the watch, for example by pressing an override button on the watch. In another embodiment, external devices may communicate with the watch as part of the medical activity to be performed by the patient. For example, a pillbox with communication capability may be configured to hold a patient's medication within. As discussed in greater detail herein, the pillbox may include sensors to determine when a portion of the pillbox (e.g., a cavity containing medication for a particular day or time) has been emptied. Upon the patient emptying this portion of the pillbox in response to a notification from the watch, the pillbox may send a confirmation message back to the watch that turns off the notifications currently being output by the watch. In an alternative embodiment, a number of different wearable items (e.g., bracelets, anklets, necklaces, eye glasses, etc.) may be used with the same functionality as the watch. In still another alternative embodiment, non-wearable items (e.g., televisions, radios, household ornaments or appliances, etc.) may be used with the same functionality as the watch.

FIG. 1B shows a side view of an electronic apparatus 170 for storing patient data and/or reminding a patient of medical activity to be performed. The electronic apparatus 170 may include features that are the same as or similar to those previously discussed for FIG. 1A. As shown, the electronic apparatus 170 may include a processor that interfaces with a display 172 (e.g., the face of a watch) for displaying information to the user, such as notifications when a user is supposed to perform a particular medical activity (e.g., take a scheduled medication). Memory of the electronic apparatus 170 interfaces with the processor for setting up the times desired for such notifications. A reset button 174 may be included to allow a user to cancel a notification (e.g., an audible notification) and to trigger the electronic apparatus 170 to prepare for notifying the user of the next medical activity in sequence that has been programmed. A USB connection 176 (e.g., a female port with a rubber stopper) is also included to allow a user to view, transfer, or update data and/or programming of the electronic apparatus 170. A night vision button 178 may be included such that, upon pressing by the user, portions of the electronic apparatus 170 illuminate to allow the user to see better in low-light conditions.

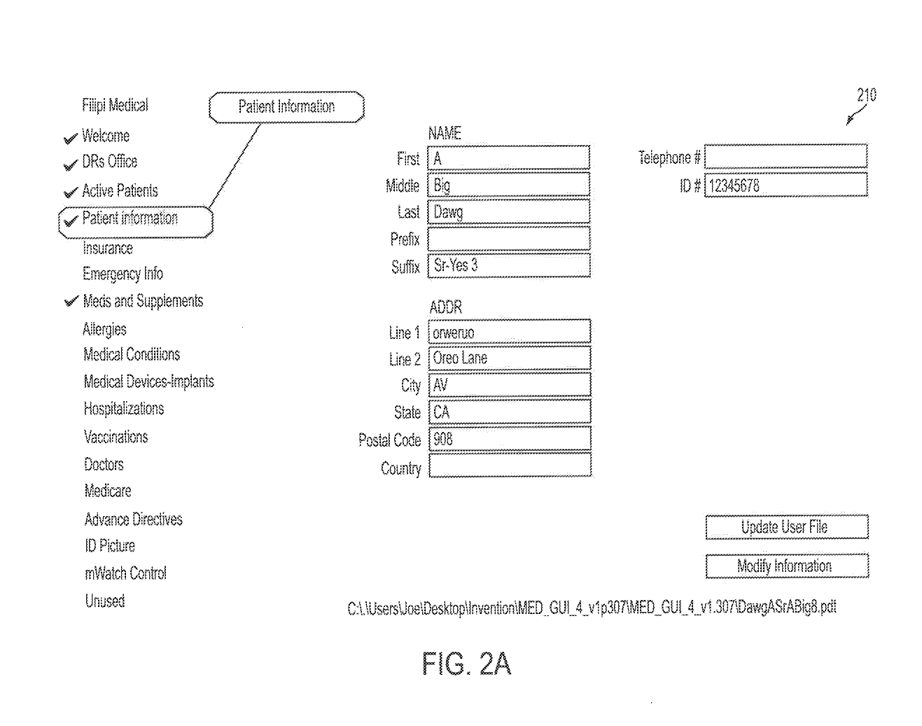

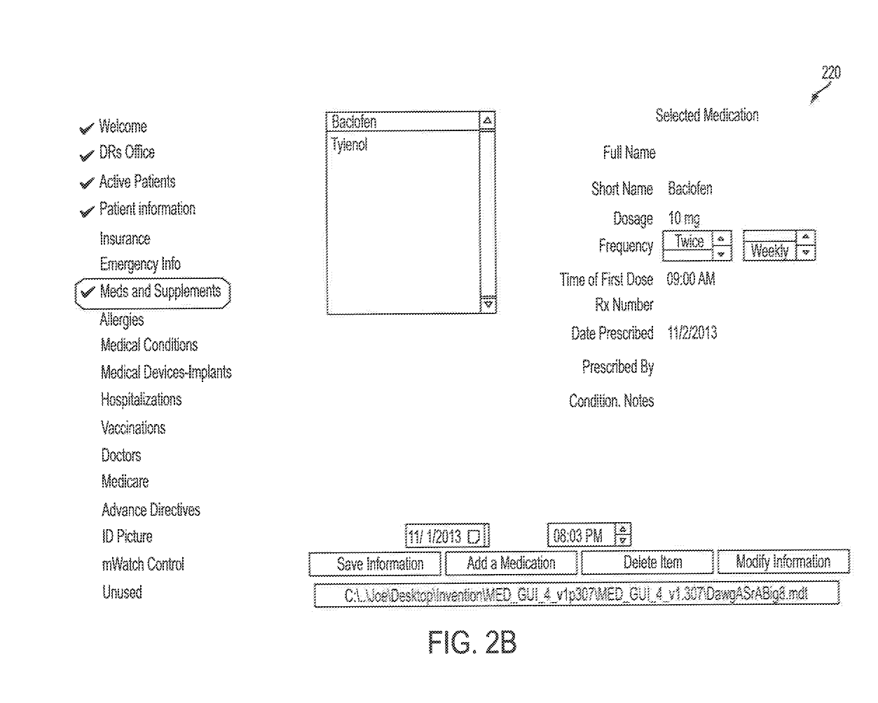

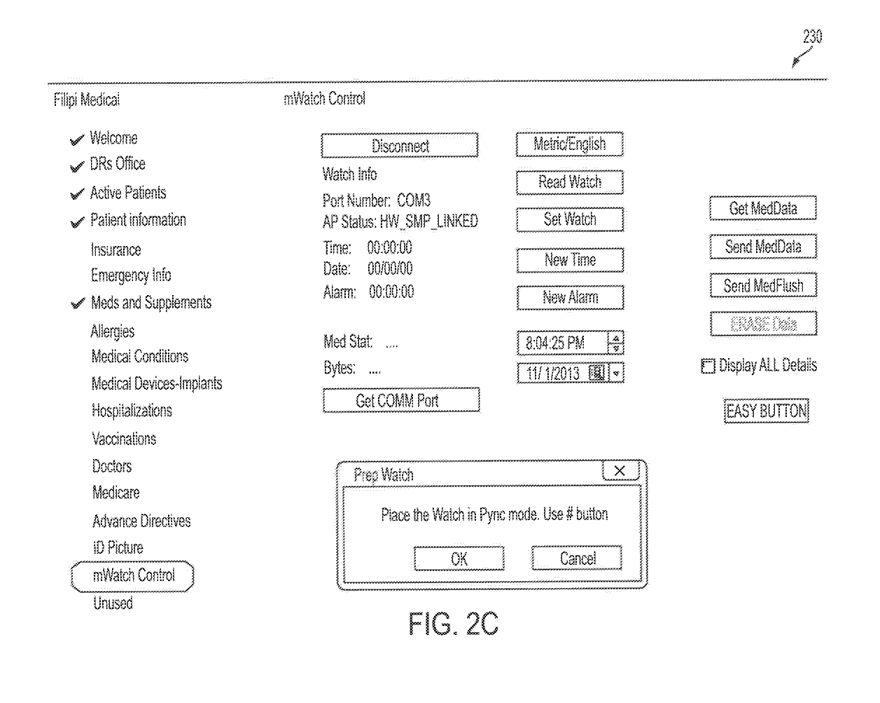

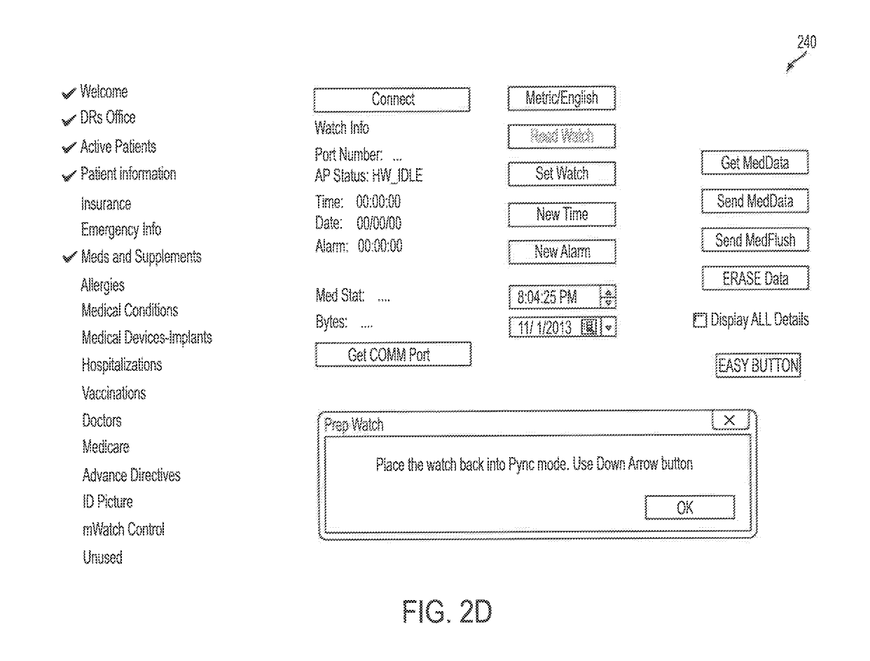

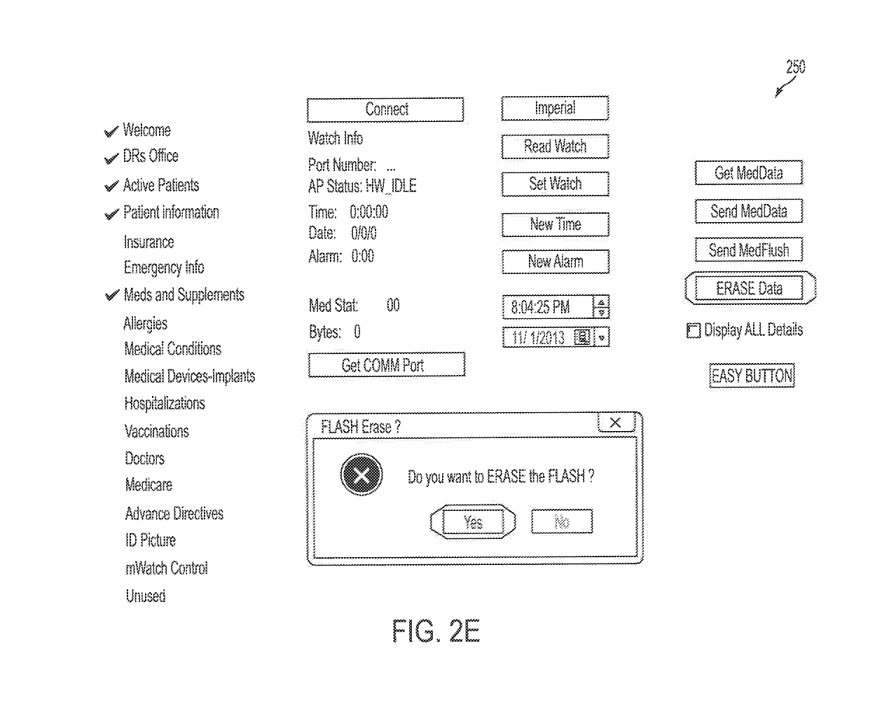

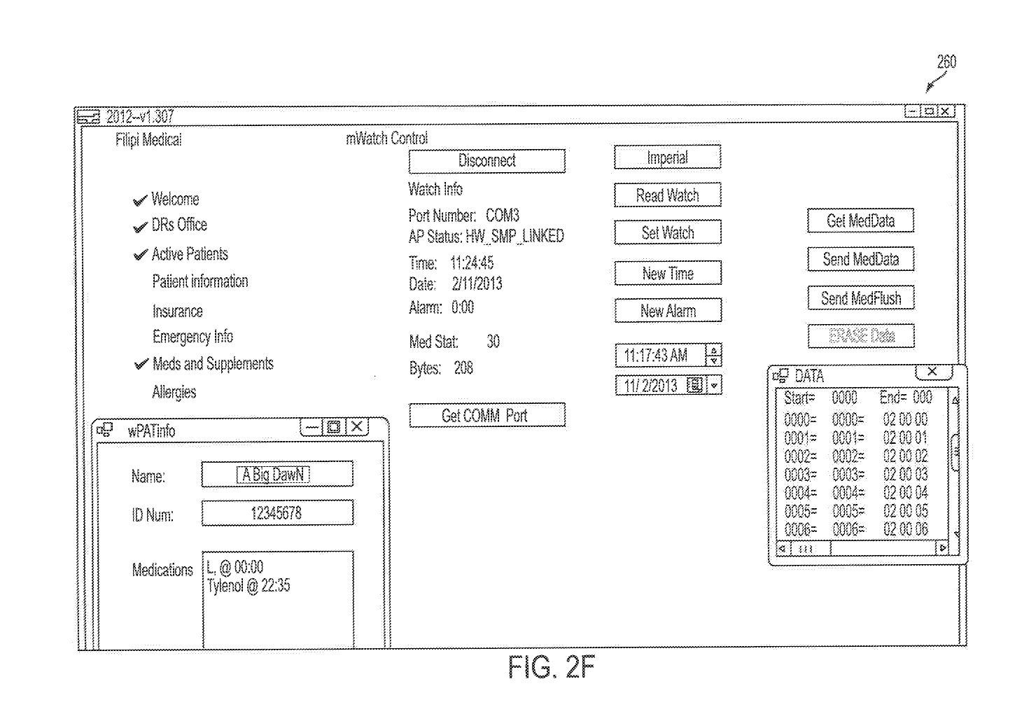

Various forms of software that run on desktop PCs, laptops, tablets, phones, etc. may be used to program and/or view information of an electronic apparatus or device, such as a watch. FIG. 2A shows a first screen of a software program configured to interface with an electronic apparatus for storing patient data and/or reminding a patient of medical activity to be performed. As shown, this screen may include menu options for allowing a user to enter particular types of information. In FIG. 2A this information is shown as patient information, such as name, address, telephone number, etc.

FIG. 2B shows a second screen of a software program configured to interface with an electronic apparatus for storing patient data and/or reminding a patient of medical activity to be performed. As shown, this screen may include information regarding medications for which a patient desires reminder notifications to help ensure compliance with the medication schedule. Changes can be made to the time, medication type, dosage, etc. In this fashion, the electronic apparatus can be initially setup and/or modified later to correspond to a particular patient's medication or other medical activity schedule for which notifications are desired to help ensure compliance.

FIGS. 2C-2F show third, fourth, fifth, and sixth screens, respectively, of a software program configured to interface with an electronic apparatus or device, such as a watch, for storing patient data and/or reminding a patient of medical activity to be performed. In this screen, the electronic apparatus may be placed into a mode or configuration such that a user can setup certain settings for the watch, such as date, time, and alarms. This mode or configuration may also allow a user to view electronic apparatus information, such as data or settings stored in the memory of the electronic apparatus. In certain embodiments, to provide simplified menus or screens to patients while still allowing physicians, care givers, or other, more advanced users access to full functionality, an “easy program” button may be provided. Upon pressing this button the screen or settings may update (see FIG. 2D) to eliminate and/or change certain of the more confusing or advanced options. FIG. 2E shows a user selecting an ERASE Data button in order to erase all or a portion of the memory (e.g., flash memory) of the electronic apparatus. FIG. 2F shows a screen where a user may press a “Get MedData” button to get medical data from the electronic apparatus, then a “Send MedData” button to transmit the data to an external and/or remote location or device, then a “Send MedFlush” button to transmit additional data (e.g., a video) to the external and/or remote location or device, and finally a “Disconnect” button to disconnect from the electronic apparatus. Although shown using specific buttons or other user-interface elements, software configured to interact with the electronic apparatus may take any of a variety of forms and utilize any of a variety of user interfaces to permit a user to program or otherwise interact with the electronic apparatus in an alternative embodiment.

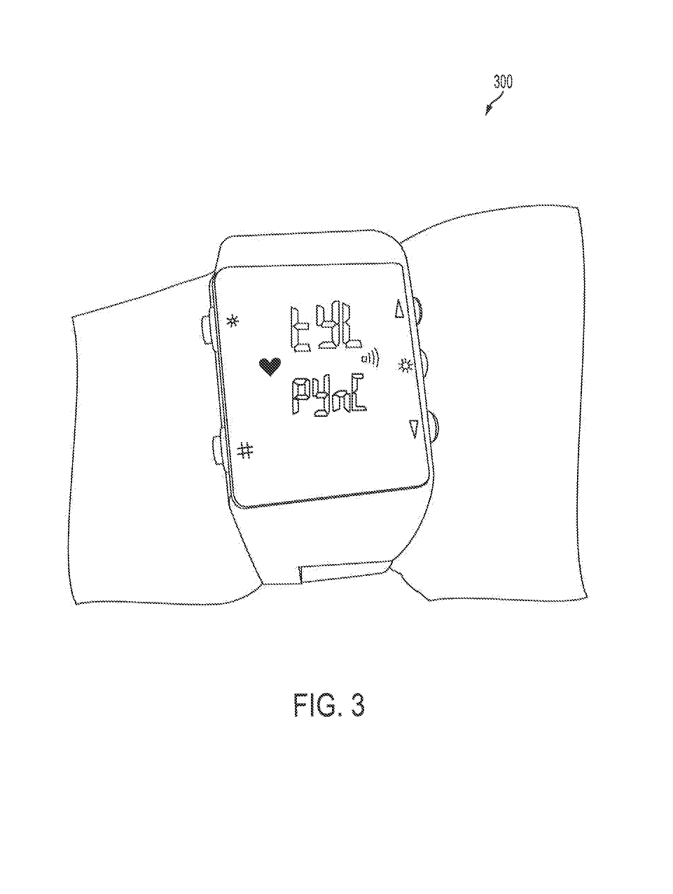

Turning next to FIG. 3, a front view of an electronic apparatus being worn by a patient that is configured to store patient data and/or remind the patient of medical activity to be performed. The electronic apparatus may be the same as or similar to those previously discussed. For example, one notification may include scrolling the name of a medication (e.g., Tylenol) across a screen of the electronic apparatus when a processor of the electronic apparatus determines that it is time for a patient to take the prescribed medication.

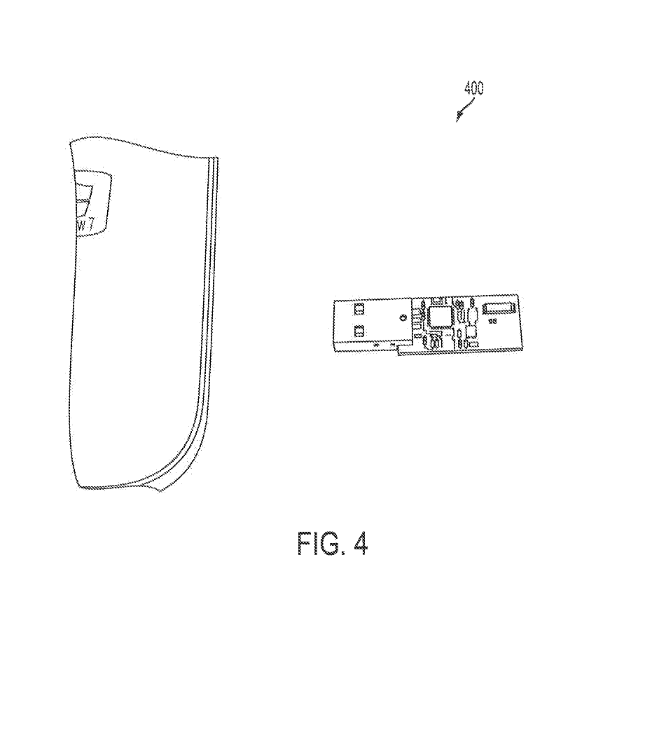

FIG. 4 shows a portable electronic receiver that is configured to interface with an electronic apparatus that is configured to store patient data and/or remind a patient of medical activity to be performed. The electronic apparatus may be the same as or similar to those previously discussed. This electronic receiver 400 may have a USB port such that it can easily be plugged in to a variety of electronic devices for communication with the electronic device. In this fashion, the portable electronic receiver 400 may be carried around by a physician, care giver, or other individual such that they can always have access to one or more electronic apparatus, no matter their geographic location.

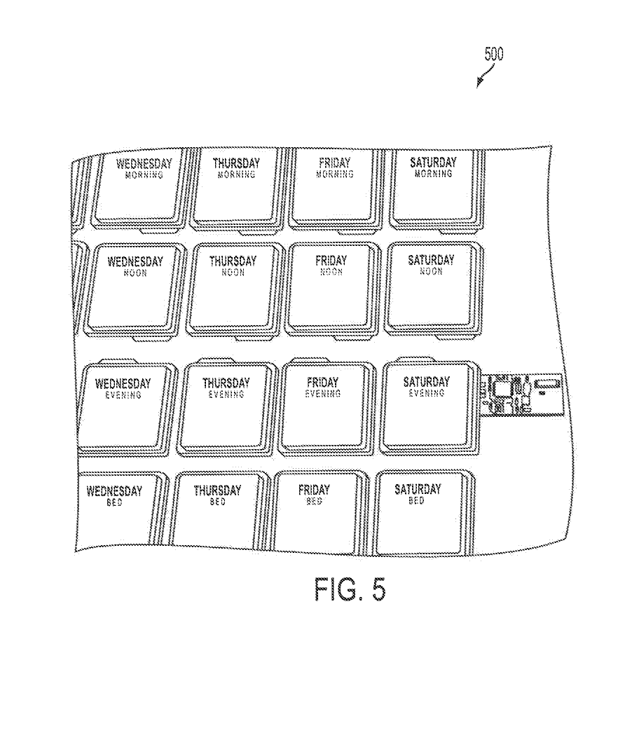

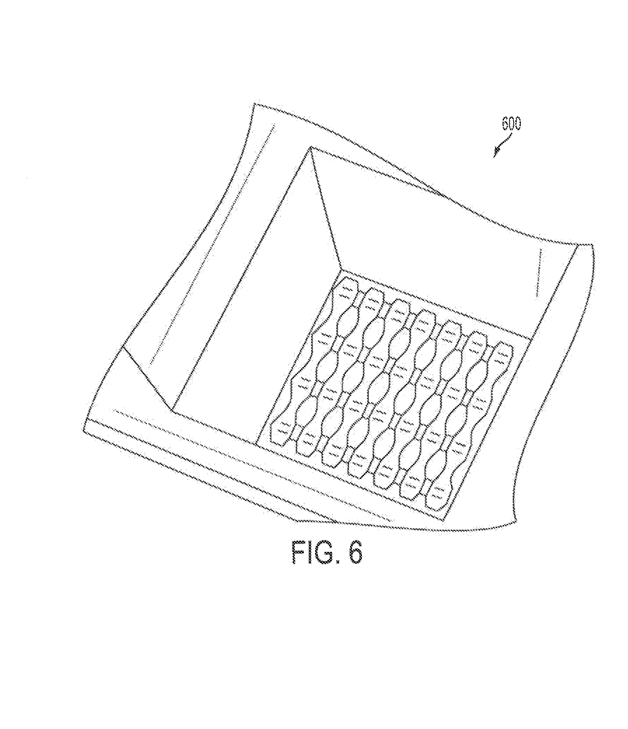

FIG. 5 shows a pillbox 500 configured to remotely interface with an electronic apparatus that is configured to store patient data and/or remind the patient of medical activity to be performed. This pillbox 500 may include features or functionality that is the same or similar to that previously discussed. In one embodiment, the pillbox 500 may be configured to communicate with a remote electronic apparatus in order to indicate to the electronic apparatus when a patient has emptied the contents of at least a portion of the pillbox 500. For example, if the electronic apparatus indicates that a user needs to take medication on Saturday evening and sounds an audible, visual, or tactile alarm or notification, upon emptying the medication in the “Saturday evening” portion of the pillbox 500, the pillbox 500 may transmit a signal back to the electronic apparatus that causes the alarm or notification to turn off. One or more sensors 600 (see FIG. 6) may be disposed within or on the pillbox 500 to help determine when contents have been emptied

In order to help encourage patient compliance, patients may participate in a program that awards points for successful compliance of the notifications of an electronic apparatus. The electronic apparatus may be the same or similar to those previously discussed. For example, upon successfully emptying the designated portion of a pillbox after a notification by the electronic apparatus, an account for the user may be updated with a particular number of points. Unsuccessful compliance by the patient (e.g., not taking the medication or delaying too long in responding to the notification to take the medication) would result in a lessened number of points, no points at all, and/or a deduction of points. The points may accumulate for the patient and later be used for rewards (e.g., products and/or services that may be bought by the patient with the accumulated points, such as items, movie tickets, jewelry, restaurant vouchers, etc.). Such rewards would be financed via a service charge by the companies wishing to participate in the rewards program.

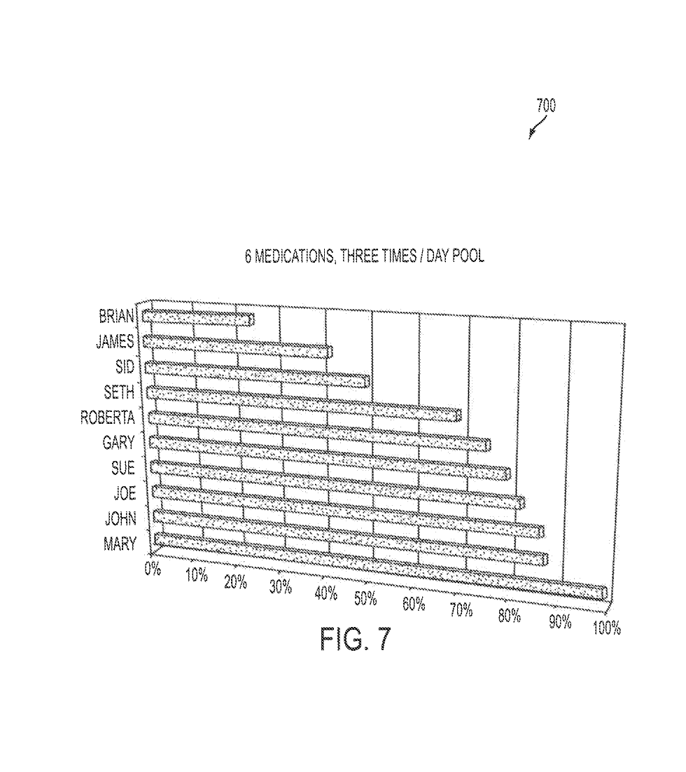

Certain embodiments of the program that awards points for successful compliance may encourage competition between patients to be the most compliant. For example, patients may be able to view their ranking or a dashboard that indicates how compliant they are when compared against similarly situated patients. FIG. 7 shows a sample collection of data 700 relating to patient compliance for the purposes of gamification of the use of the electronic apparatus. As shown, different “pools” may be setup based upon the amount of medication or other medical activity that a patient is to be notified about, for example, in a given day. Thus, patients who take six medications, three times a day may be placed into a different pool than patients who only take three medications, once a day. In this fashion, competition between patients is fairer since patients with multiple notification reminders in a day would only be competing for a compliance score with other patients that are similarly seeing multiple notification reminders. In certain embodiments, in addition to providing encouragement for compliance via competition, patients may receive additional points and/or rewards based upon their ranking versus other patients. For example, each predetermined time period (e.g., week, month, etc.) a winner may be selected for highest compliance rate in a given pool. Compliance could be measured from the time an alarm or notification is discharged from the electronic apparatus to the time a patient complies (e.g., by emptying the pill box). For example, if the pillbox chamber is emptied within a 5 minute window, the compliance could be measured at 100%. However, if the pillbox chamber was emptied outside of the 5 minute window, but within the 10 minute window, the compliance could be measured at 95%. Different points could be awarded to the patient based upon the level of compliance.

In certain embodiments, in order to facilitate the notification system and/or points and/or rewards gamification of use of the electronic apparatus, data or information may be transmitted and/or viewable at remote locations. Doctors or care givers may be able to access the compliance rankings and/or other information regarding patient compliance to determine the best possible treatment for a particular patient. For example, if a doctor notices that a patient is regularly non-compliant in the afternoon hours, the doctor may alter the patient's prescribed medical activity to only include a morning and/or evening schedule. In another example, if a patient does not comply with a notification within a first predetermined time, a message (e.g., an SMS text message) may be transmitted out to a previously designated person. If, after a second predetermined time, the patient still has not complied, a message (e.g., SMS text message) to a call center. In such a case, the noncompliant patient may receive a phone call from staff, doctor, family member or other individual who was contacted by the call center.

The previous description of the disclosed examples is provided to enable any person of ordinary skill in the art to make or use the disclosed methods and apparatus. Various modifications to these examples will be readily apparent to those skilled in the art, and the principles defined herein may be applied to other examples without departing from the spirit or scope of the disclosed method and apparatus. The described embodiments are to be considered in all respects only as illustrative and not restrictive and the scope of the invention is, therefore, indicated by the appended claims rather than by the foregoing description. All changes which come within the meaning and range of equivalency of the claims are to be embraced within their scope. Skilled artisans may implement the described functionality in varying ways for each particular application, but such implementation decisions should not be interpreted as causing a departure from the scope of the disclosed apparatus and methods. The steps of the method or algorithm may also be performed in an alternate order from those provided in the examples.

What is claimed is:

1. An watch for a patient comprising:
a band configured to be worn around a wrist of the patient;
a processor coupled with the band; and
a screen coupled with the band and the processor,
wherein the screen is configured to display a message relating to a medical activity to be performed by the patient.

2. The watch of claim 1 wherein at least a portion of the band or screen is configured to illuminate if the message is displayed.

3. The watch of claim 1 further comprising a memory coupled with the processor, the memory configured to store medical information about the patient.

4. The watch of claim 1 further comprising a speaker coupled with the processor, the speaker configured to sound an alarm relating to the medication to be taken by the patient.

5. The watch of claim 4 wherein the medication to be taken by the patient is a blood sugar check reminder.

6. The watch of claim 1 further comprising a USB port coupled with the band, the USB port configured to allow access to the medical information about the patient stored on the memory.

7. The watch of claim 1 wherein the processor is configured to communicate with a second device via Bluetooth technology.

8. The watch of claim 7 wherein the second device is chosen from the group consisting of a watch, a pillbox, a transmitter located in the patient's home, a remote call center, and a transmitter located in a physician's office.

9. A medication notification system comprising:
a watch having:
a wristband,
a speaker,
a display, and
a processor connected with the display, the processor configured to cause the speaker to alarm based on a schedule for consumption of a medication; and
a pillbox having a processor, the processor of the pillbox configured to interface with the processor of the watch,
wherein the speaker ceases to alarm based on at least a portion of the pillbox being emptied.

10. The medication notification system of claim 9 further comprising a remote device configured to communicate with the processor of the watch, the remote device configured to program the processor.

11. The medication notification system of claim 9 wherein the predetermined time is programmable by a user of the medication notification system.

12. The medication notification system of claim 11 wherein at least a portion of the wristband of the watch is configured to illuminate red if the speaker is caused to alarm until the at least a portion of the pillbox is emptied.

13. The medication notification system of claim 12 wherein the at least a portion of the wristband of the watch is configured to illuminate green if the at least a portion of the pillbox is emptied.

14. The medication notification system of claim 13 wherein the speaker emits a success sound if the at least a portion of the pillbox is emptied.

15. A method for encouraging compliance of a patient for a medical activity comprising:
providing a watch to the patient;
programming the watch to notify the patient about the medical activity;
notifying the patient about the medical activity via the watch;
determining whether the patient complied with the notifying of the patient about the medical activity; and
transmitting information corresponding to whether the patient complied with the notifying of the patient about the medical activity.

16. The method of claim 15 wherein the step of transmitting information includes transmitting the information to the physician of the patient.

17. The method of claim 16 wherein the watch includes an radio frequency identification (RFID) tag.

18. The method of claim 15 further comprising updating the programming of the watch.

19. The method of claim 17 wherein the step of updating the programming of the watch includes connecting an external device to the watch via a USB connection.

20. The method of claim 17 wherein the step of updating the programming of the watch includes wirelessly connecting an external device to the watch via BlueTooth technology.
